(19) United States
(12) Patent Application Publication
(10) Pub. No.: US 2021/0388078 A1
(43) Pub. Date: Dec. 16, 2021

(54) Novel method and compounds for treatment of cognitive loss associated with adult onset leukodystrophy with axonal spheroids and pigmented glia (ALSP) and other neurodegenerative diseases involving reduced colony stimulating factor-1 receptor (CSF-1R) signaling

(71) Applicant: Albert Einstein College of Medicine, Inc, Bronx, NY (US)

(72) Inventors: Richard E. Stanley, New York, NY (US); Violeta Chitu, Scarsdale, NY (US)

(21) Appl. No.: 17/284,599

(22) PCT Filed: Oct. 13, 2019
(86) PCT No.: PCT/US19/56019

§ 371 Date: Apr. 12, 2021

Related U.S. Application Data
(60) Provisional application No. 62/745,304, filed on Oct. 13, 2018.

(51) Int. Cl. C07K 16/24; A61K 31/7088; A61P 25/28

(52) U.S. Cl. CPC C07K 16/243; A61P 25/28; A61K 31/7088

(57) ABSTRACT

Adult-onset leukoencephalopathy with axonal spheroids and pigmented glia (ALSP) is caused by dominant inactivating mutations in the colony stimulating factor receptor 1 (CSF1R) kinase domain. GM-CSF haploinsufficiency corrects olfactory, cognitive and emotional functions lost in Csf1r+/− mice. This correlates with the correction of microgliosis and microglial functions resulting in improvement of myelination and rescue of neurogenesis. However, GM-CSF haploinsufficiency fails to correct the motor deficits of Csf1r+/− mice and cerebellar microgliosis. The present invention discloses methods and compositions using GM-CSF as a suitable therapeutic target to inhibit in amelioration of the cognitive impairments in ALSP and other in conditions involving inflammatory activation of microglia and macrophages, such as AD, ALS, multiple sclerosis, and hippocampal inflammation following radiation therapy. Treatment with GM-CSF inhibitors is beneficial in ALSP, as adult neurogenesis is important for memory, olfaction and prevention of anxiety/depression and early initiation of such treatment in carriers of CSF1R mutations may increase effectiveness. Balancing the actions of CSF-1R and GM-CSF signaling are necessary to preserve olfaction, cognition and emotional balance in aged mice. This balance is likely altered in many neurodegenerative diseases in which activated microglia contribute to the pathology.

### CROSS REFERENCE TO RELATED APPLICATIONS

This application is the US national application of PCT/US 2019/056019, filed on 13 Oct. 2019 and which claims benefit of and priority to U.S. Provisional Patent Application No. 62/745,304, filed on 13 Oct. 2018, now expired, the disclosure of which is incorporated by reference in its entirety.

This invention was made with Government support under Grant Number R01NS091519 awarded by the National Institutes of Health, The Government has certain rights in the invention.

### BACKGROUND OF THE INVENTION

### 1. Field of the Invention

The present invention relates to the field of cognitive loss associated with adult onset leukodystrophy with axonal spheroids and pigmented glia (ALSP) and other neurodegenerative diseases involving reduced CSF-1R signaling.

### 2. Description of the Prior Art

Adult-onset leukoencephalopathy with axonal spheroids and pigmented glia (ALSP) is caused by dominant inactivating mutations in the CSF1R kinase domain. There is no effective treatment for ALSP. Without post-mortem examination, brain biopsy or genetic testing, ninety percent of ALSP patients have been misdiagnosed as having Alzheimer's disease (AD).

Previous studies from our laboratory have shown that the CSF-1R is required for the development and maintenance of all microglia (brain macrophages). In other studies, we have shown that CSF-1 suppresses the inflammatory state of macrophages and identified a major mechanism. Furthermore, CSF-1 overexpression in brain has been shown to attenuate inflammatory activation of microglia. In contrast, GM-CSF enhances the inflammatory activation of microglia and macrophages and, in a model of multiple sclerosis, has been shown to promote monocyte migration across the blood-brain barrier and to trigger an inflammatory signature in infiltrating monocytes. The present invention is based on several findings:

1) GM-CSF, the other major CSF-1R-independent microglial mitogen, is increased along with microglia in young ALSP mice prior to development of disease.
2) As older mice develop disease, microglial numbers remain elevated and other markers of inflammatory microglia appear, including several GM-CSF target genes.
3) Deletion of a single GM-CSF allele prevents the loss of cognition, olfactory deficits and depression.
4) Monoallelic deletion of the GM-CSF gene normalizes the microgliosis, the decreased callosal volume and the decreased adult neurogenesis. It also improves myelination.
5) ALSP mouse microglia exhibit changes in gene expression, consistent with neurotoxic responses and with mitochondrial dysfunction causing oxidative stress. Deletion of a single GM-CSF allele restores most of these gene expression changes and decreases oxidative stress.
6) Microglial CSF-1R expression is decreased in other neurodegenerative diseases, including AD and amyotrophic lateral sclerosis (ALS) and there is significant overlap between the set of genes dysregulated in ALSP and rescued by deletion of a single GM-CSF allele and those dysregulated in AD and ALS.

This invention is based on novel information obtained by the inventors and not found in the prior art, using a mouse model of ALSP that is also incorporated in this disclosure. Furthermore, although a rare disease, studies of ALSP are relevant to several neurodegenerative diseases. The inventors discovered that any disruption of the normal balance of the quiescent (CSF-1R regulated) and activated state (GM-CSFR regulated) states of microglia cause neurocognitive impairment and that re-establishing this balance prevents the cognitive decline.

### SUMMARY OF THE INVENTION

The present invention provides methods and compositions for the treatment of cognitive loss in several neurodegenerative diseases through the identification of granulocyte-macrophage colony stimulating factor (GM-CSF) signaling as an appropriate target to prevent loss of cognitive function in ALSP and other neurodegenerative diseases.

The present invention further discloses methods and compositions for the treatment of patients with other diseases associated with microgliosis and neuro-inflammation, such as but not limited to multiple sclerosis.

Further, the present invention creates a platform technology based on the identification of a signaling pathway that can be therapeutically targeted by a variety of means to inhibit loss of cognitive functions in ALSP and other neuro-inflammatory conditions. These would include bispecific antibodies to GM-CSF that cross the blood brain barrier (BBB), anti-sense oligonucleotides, small molecules that inhibit GM-CSF signaling and cross the BBB and small molecules that activate CSF-1R signaling and cross the BBB.

Targeting GM-CSF in adults avoids noticeable side effects since GM-CSF—null mice survive quite well and the major defects are primarily developmental in origin and blockade need not be total.

The development of novel compounds targeting GM-CSF would offer additional therapeutic options in the treatment of ALSP, AD, ALS, MS and cognitive impairments.

The present invention further describes a mouse model and methods used for testing anti-GM-CSF signaling therapies.

### BRIEF DESCRIPTION OF DRAWINGS

FIG. 1: Increased microglia in the brains of Csf1r+/− male mice. Iba1 staining of microglia in different areas of the brain of 11-week and 10-month-old mice. Lower panels, quantification of microglia in prefrontal and hippocampal sections. Grey bars, Csf1r+/+, Green bars, Csf1r+/+. Mean+/−S.E.M. n=3, *p<0.05 compared to Csf1r+/+ controls. Student's two-tabled t test. M1, primary motor cortex, M2, secondary motor cortex.

FIG. 2: Csf1r haploinsufficiency-associated changes in the expression of mRNAs of Inflammatory cytokines, chemokines and receptors identified by qRT-PCR in the anterior motor cortex and corpus callosum at 7 weeks (A) and 12 months (B) of age. Beta actin and GAPDH were used as housekeeping gene standards in all experiments. Means +/−S.E.M., n=3 mice/genotype. The two sided moderate t-test was performed using the LIMMA package in Bioconductor while accounting for batch effects where appropriate,*, p<0.05 vs Csf1r+/+, Grey bars indicate Csf1r+/+, green bars indicate Csf1r+/−.

FIG. 3: Prevention of loss of spatial memory in ALSP (Csf1r+/−) mice by deletion of a single Csf2 (GM-CSF) allele. A, Lower preference for the relocation object by 14.5-month old Csf1r+/− mice is corrected in Csf1r+/−: Csf2+/− mice [F(3,94)=9.758, p<0.0001]. Discriminatory ratio is the percentage of time spent exploring the relocated object over the time spent exploring both relocated and fixed objects after a 25 min interval. B, Lower preference for the novel object by 7-month old Csf1r+/− mice is corrected in Csf1r+/−; Csf2+/− mice [Pearson Chi-Square=13.644, p=0.0034]. Filled areas, fraction of mice spending more than 55% of the time exploring the novel object after a 60 min interval (mouse numbers in bars). C, Lower preference for the novel object by 17-month old Csf1r+/− mice is corrected in Csf1r+/−; Csf2+/− mice [Two-way ANOVA; interaction (object, genotype) F(3, 82=3.886, p=0.0119)); genotype, F(3, 82=0.08021, p=0.9706)]. Time exploring the novel and familiar object after a 24 h interval. D, Lower preference for the novel arm of a Y-maze by 13.5-month-old Csf1r+/− mice is corrected in Csf1r+/−; Csf2+/− mice. Percentage of entries into the novel and known arms of the maze after a retention interval of 1 h. *, p<0.05: **, p<0.01;***, p<0.001;****, p<0.0001.

FIG. 4: CSF haploinsufficieny restores responses of Csf1r+/− mice to pure odorants. (A) Odor discrimination. Attraction to explore vanilla.)B) Odor threshold to 2-phenylethanol.

FIG. 5: GM-CSF heterozygosity rescues depression in male but not motor coordination deficits in female Csf1r+/− mice. (A) increased depression-like behavior in male Csf1r+/−r mice is corrected in Csf1r+/−; Csf2r+/− mice. (B, C) the locomotor coordination deficit of Csf1r+/− mice is female-specific and is not rescued by Csf2 heterozygosity. (B) Locomotor coordination in 14-month-old female and male mice assessed as number of slips in the balance beam test. (C) Ataxia score in 13-month-old female and male mice assessed as sum of the ledge, hind limb and gait scores. For all multiple comparisons,*, p<0.05;**, p<0.01 and ***, p<0.0001.

FIG. 6: GM-CSF haploinsufficiency attenuates the neurocognitive impairment of aged ALSP mice. Neurocognitive impairment scores determined from the analysis of 10 behavioral tests. In each behavioral test, mice whose scores fell within 2 standard errors of the mean [95% confidence limits] of the average wt values were considered to “pass” and assigned a score of 0. those falling outside this interval were considered to ‘fail’ and assigned a score of 1. the neurocognitive implement score is the total number of failed tests [n=10]. The results were from the following tests. forced swim (8 months). olfactory detection dysfunction (8 months). Olfactory threshold dysfunction (9 months). Ataxia Score (12 months). Balance beam (13 months), Y Maze (13 months). Object recognition (7 months), Object placement (14 months). Morris water maze training (17 months] and Fear conditioning (content 18 months)*, significantly different, p<0.05.

FIG. 7: The decrease in callossal volume in 18 month-old Csf1r+/− mice is reversed by GM-CSF haploinsufficiency. Callossal volume determined from by MRI (n=5-8 mice per group, p<0.05).

FIG. 8: GM-CSF haploinsufficiency normalizes microglial densities in the cerebrum, but not the cerebellum of Csf1r+/− mice. (A) M1 motor cortex of 11-month old mice. (B) different brain regions of 18-month-old mice. (dhet Csf1r+/−; Csf2r+/−, *, p>0.05).

FIG. 9: GM-CSF ameliorates the neurogenic decline of older Csf1r+/− mice. Hippocampal double cortin+(DCX+) neuroblast densities in 18-month-old mice.*, p<0.05.

### DETAILED DESCRIPTION

In the following detailed description, numerous specific details are set forth in order to provide a thorough understanding of the present invention. However, well-known methods, procedures, systems, and components have not been described in detail so as not to unnecessarily obscure aspects of the various embodiments.

ALSP is a rare, autosomal dominant, neurodegenerative disorder characterized by adult-onset dementia with motor impairment and epilepsy and death within 5-7 years. ALSP is caused by inactivating mutations of the CSF1R gene, in the region encoding the intracellular kinase domain.

The CSF-1R is regulated by two cognate ligands, CSF-1 and interleukin-34 (IL-34) and is expressed on microglia, neural progenitor cells (NPCs) and several neuronal subtypes. It is required for the development and maintenance of all microglia. In addition, CSF-1 or IL-34 activation of the CSF-1R on NPCs suppresses NPC self-renewal and stimulates neuronal survival and differentiation. Furthermore, brains of Csf1r−/−nullizygous mice and humans with homozygous CSF1R mutations have gross anatomical and histological abnormalities that affect areas (cortex, corpus callosum) disrupted in ALSP.

The CSF1R mutations first described in ALSP families, included missense mutations affecting highly conserved residues and splice-site mutations leading to in-frame deletions. Furthermore, the discovery of an ALSP patient with a CSF1R frame-shift mutation that abolished protein expression proved that CSF1R haploinsufficiency is sufficient to cause ALSP.

Since the initial report of inactivating mutations in man could be explained by haploinsufficiency, the inventors of the present application studied Csf1r+/− mice and showed that these mice exhibit behavioral, radiologic, histopathologic and ultrastructural alterations associated with neuronal degeneration and microgliosis, similar to the changes observed in ALSP patients.

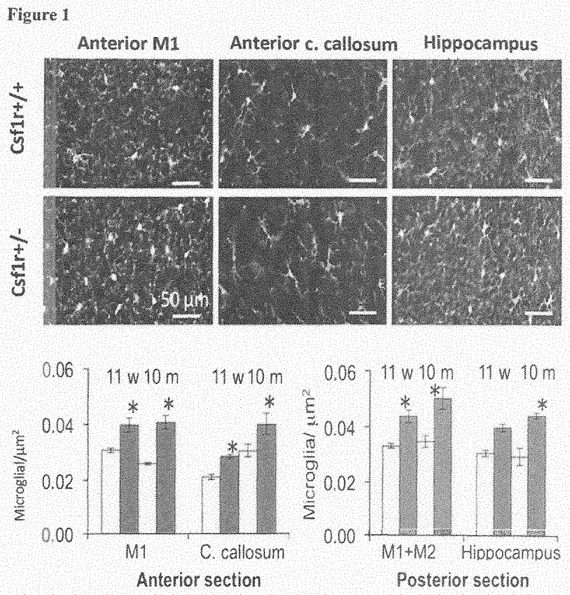

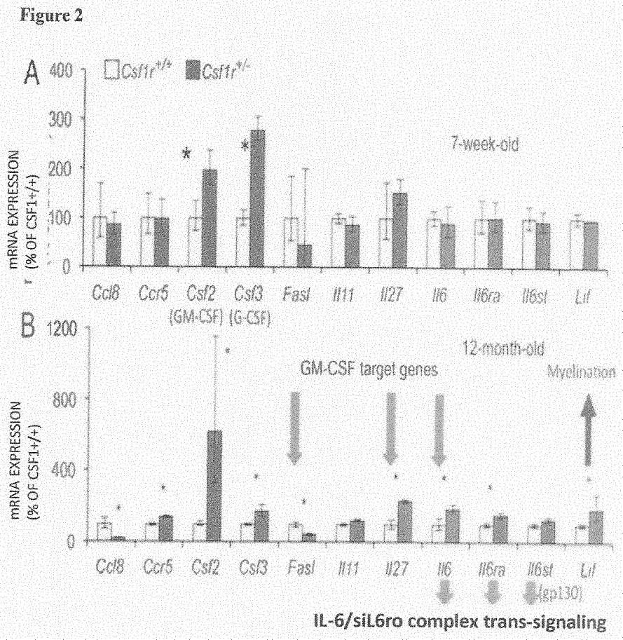

Relevant to this invention, the present inventors showed that both young and old Csf1r+/− mice had elevated densities of microglia in various brain regions (FIG. 1). The increase in microglia in young mice was not associated with a compensatory increase in either of the CSF-1R ligands, but was instead associated with an increase in the expression of GM-CSF, an inflammatory cytokine and the only other powerful microglial mitogen, and of G-CSF (FIG. 2A). In older mice, besides the elevation these two cytokines, the expression of other known markers of inflammation, including several GM-CSF target genes, were altered as expected in inflammation (FIG. 2B). Their additional previous studies of demonstrated that CSF-1R signaling suppresses the expression of inflammatory mRNAs in macrophages, consistent with the possibility that Csf1r+/− microglia increased their expression of GM-CSF due to their decreased CSF-1R signaling. The inventors therefore investigated whether removal of a single GM-CSF allele corrected the behavioral, structural and histological abnormalities in the Csf1r+/− mouse model of ALSP.

### Experimental Results Underpinning the Basis of the Invention

### GM-CSF Haploinsufficiency Restores Cognitive, Emotional and Olfactory Functions to Csf1r+/− Mice.

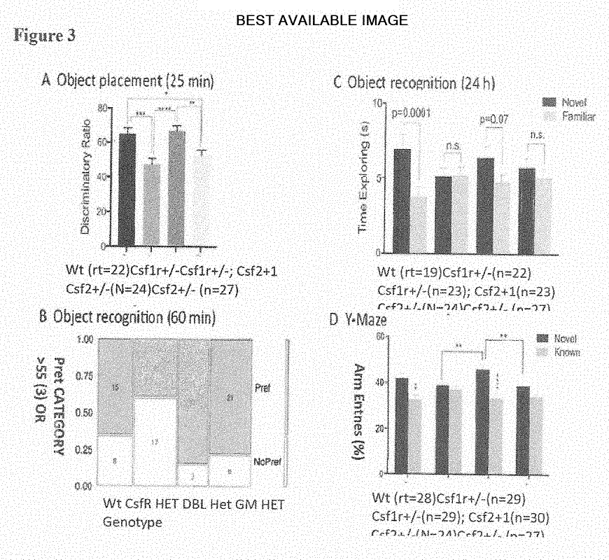

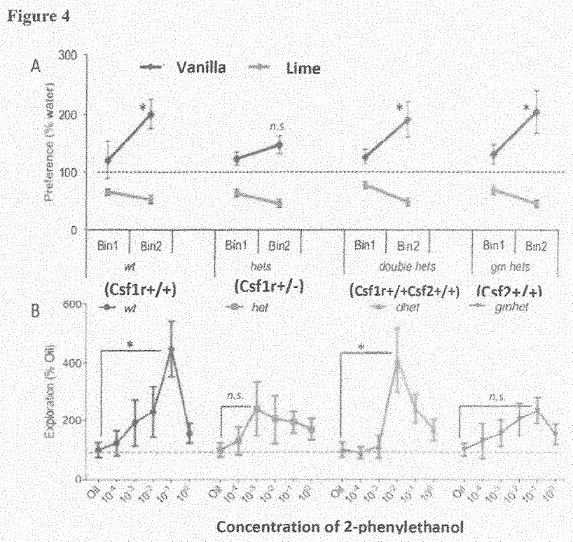

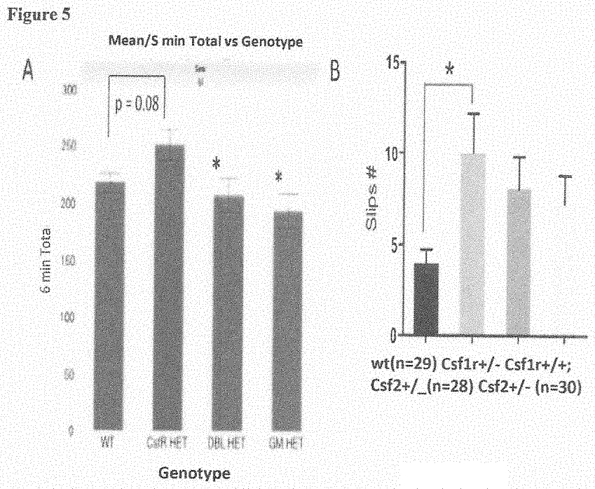

To test whether deletion of a single GM-CSF allele in Csf1r+/− mice prevented their development of these deficits, the inventors examined the behavior of large cohorts of male and female mice. Rescue of the impairment of spatial memory in Csf1r+/− mice by removal of a single GM-CSF allele was demonstrated in four different tests (FIG. 3). In addition, GM-CSF haplo-insufficiency restored responses of Csf1r+/− mice to pure odorants (FIG. 4). The inventors have previously shown that male, but not female Csf1r+/− mice, exhibit depressive behavior. As shown in FIG. 5A, depression in male Csf1r+/− mice was also rescued by removal of a single GM-CSF allele. However, GM-CSF haploinsufficiency failed to rescue motor deficits of Csf1r+/− mice, assessed by their performance on the balance beam (FIG. 5B) and by their ataxia score, calculated from separate measurements of hind limb clasping, ledge crawling and gait (data not shown).

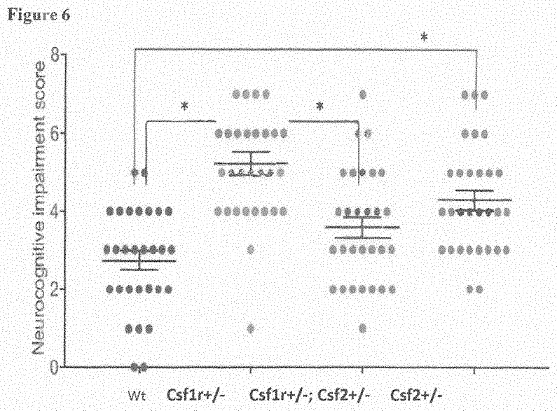

Summarizing the performance of mice in all these multiple tests, GM-CSF haploinsufficiency attenuated the neurocognitive impairment in aged Csf1r+/− mice (FIG. 6).

### GM-CSF Haploinsufficiency Restores Callosal Volume in Csf1r+/− Mice:

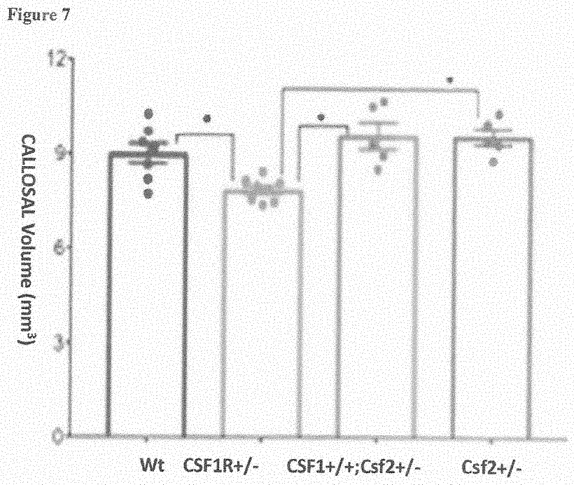

ALSP patients exhibit callosal atrophy that can occur as early as 5 years before disease onset. The inventors have previously shown that aging Csf1r+/− mice also exhibit a decreased thickness of the corpus callosum (Chitu et al., 2015). Analysis of affected mice (FIG. 7) demonstrated that the callosal volume of Csf1r+/− mice was decreased and that this was normalized by deletion of a single GM-CSF allele. GM-CSF haploinsufficiency normalizes microglial densities in the cerebrum, but not the cerebellum, of Csf1r+/− mice:

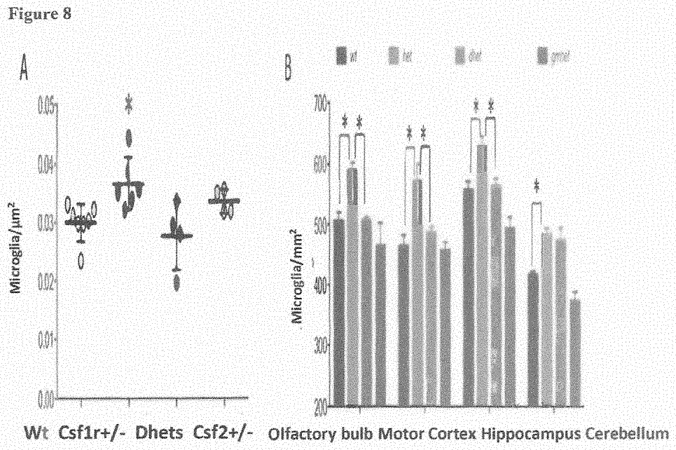

Microglial specific deletion of a single CSF-1R allele reproduced the increased microglial densities of young Csf1r+/− mice (data not shown) indicating a lineage autonomous triggering of the microgliosis. Deletion of a single GM-CSF allele in young Csf1r+/− mice normalized microglial densities (FIG. 8A). Similar normalizations of microglial densities by GM-CSF haploinsufficiency were found in older mice in various areas of the cerebrum, but not in the cerebellum (FIG. 8B).

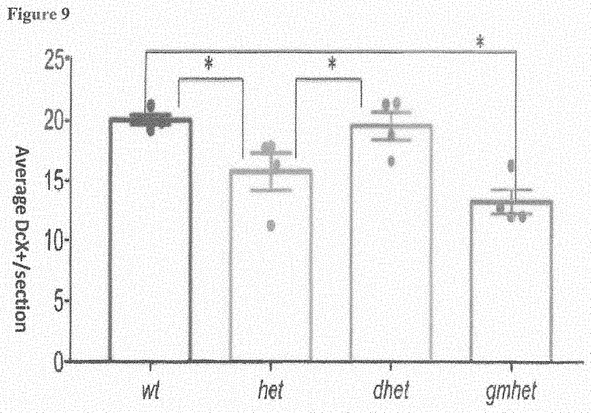

GM-CSF haploinsufficiency ameliorates the neurogenic decline of older Csf1r+/− mice: Hippocampal neurogenesis plays an important role in learning and memory. Consistent with their behavioral phenotypes, by 18 months of age, the Csf1r+/− cohort exhibited neurogenic decline in the hippocampus that was normalized by deletion of a single GM-CSF allele (FIG. 9).

### Conclusions:

GM-CSF haploinsufficiency corrects olfactory and cognitive functions lost in Csf1r+/− mice and their depressive behavior. This correlates with the correction of microgliosis in the olfactory bulb and hippocampus and with the rescue of hippocampal neurogenesis. However, GM-CSF haploinsufficiency fails to correct the motor deficits of Csf1r+/− mice.

One embodiment of the present invention provides for the use of GM-CSF as a suitable therapeutic target to inhibit in amelioration of the cognitive impairments in ALSP and other neurodegenerative conditions involving microglia and macrophages, such as AD, ALS, MS and hippocampal inflammation following radiation therapy. Treatment with GM-CSF inhibitors is beneficial in ALSP, as adult neurogenesis is important for both memory, olfaction and reduction of anxiety and depression-like behaviors and early initiation of such treatment in carriers of the mutation increases effectiveness. These would include bispecific antibodies to GM-CSF that cross the blood brain barrier (BBB), anti-sense oligonucleotides, small molecules that inhibit GM-CSF signaling and cross the BBB and small molecules that activate CSF-1R signaling and cross the BBB. The inventors discovered that balanced actions of CSF-1R and GM-CSF signaling are necessary to maintain olfactory responses and short-term memory in aged mice. This balance is likely altered in many neurodegenerative diseases in which activated microglia contribute to the pathology.

Although illustrated and described above with reference to certain specific embodiments, the present invention nevertheless is not intended to be limited to the details shown. Rather, various modifications may be made in the details within the scope and range of equivalents of the claims and without departing from the spirit of the invention.

1. A method for treatment of a subject having a neurodegenerative disease comprising attenuating activation of granulocyte-macrophage colony stimulating factor (GM-CSF).

2. The method of claim 1 wherein the disease is leukodystrophy with axonal spheroids and pigmented glia (ALSP) causing an observed impairment.

3. The method of claim 2 wherein the impairment is in cognitive function.

4. The method of claim 2 wherein the impairment is in emotional function.

5. The method of claim 2 wherein the impairment is in olfactory function.

6. The method of claim 1 wherein the disease is associated with microgliosis, decreased CSF-1R signaling, or neuro-inflammation.

7. The method of claim 3 wherein the disease is AD, ALS, or multiple sclerosis.

8. A method for preventing cognitive decline in a subject having a neurodegenerative disease comprising maintaining a normal balance between CSF-1R and GM-CSFR effects.

9. The method of claim 8 wherein the disease is leukodystrophy with axonal spheroids and pigmented glia (ALSP) causing neurocognitive impairment.

10. The method of claim 8 wherein the disease is associated with microgliosis, neurodegeneration, or neuro-inflammation.

11. The method of claim 10 wherein the disease is AD, ALS, or multiple sclerosis.
